(19) United States
(12) Patent Application Publication
(10) Pub. No.: US 2022/0152211 A1
(43) Pub. Date: May 19, 2022

(54) HYBRID CURCUMIN CONJUGATES AND METHODS OF USE THEREOF

(71) Applicants: AUGUSTA UNIVERSITY RESEARCH INSTITUTE, INC., Augusta, GA (US); THE US GOVERNMENT AS REPRESENTED BY THE DEPARTMENT OF VETERANS AFFAIRS, Washington, DC (US)

(72) Inventors: Siva S. PANDA, Martinez, GA (US); Muthusamy THANGARAJU, Evans, GA (US); Balakrishna L. LOKESHWAR, North Augusta, SC (US)

(73) Assignees: AUGUSTA UNIVERSITY RESEARCH INSTITUTE, INC., Augusta, GA (US); THE US GOVERNMENT AS REPRESENTED BY THE DEPARTMENT OF VETERANS AFFAIRS, Washington, DC (US)

(21) Appl. No.: 17/525,473

(22) Filed: Nov. 12, 2021

Related U.S. Application Data
(60) Provisional application No. 63/113,586, filed on Nov. 13, 2020.

(51) Int. Cl. A61K 47/55; A61P 35/00

(52) U.S. Cl. CPC A61K 47/55; A61P 35/00

(57) ABSTRACT

Hybrid curcumin-based conjugates and methods of use thereof are provided. Pharmaceutical compositions including an effective amount of one or more curcumin conjugates are also provided. In particular embodiments, the compositions are formulated for oral delivery. The conjugates and pharmaceutical compositions thereof can be administered to a subject in need thereof to treat cancer.

### CROSS-REFERENCE TO RELATED APPLICATIONS

This application claims priority to and benefit of U.S. Provisional Patent Application No. 63/113,586 filed on Nov. 13, 2020, and which is incorporated by reference in its entirety.

### FIELD OF THE INVENTION

This invention is generally in the field of curcumin-based drugs and methods of use thereof, particularly for treatment of cancer.

### BACKGROUND OF THE INVENTION

Breast cancer (BC) is the second most common cancer in women with an estimated 268,600 cases in 2019 and 41,760 deaths (Siegel et al., CA Cancer J Clin. 2019, 69, 7-34; American Cancer Society, Cancer Facts & Figures 2019). BC is a complex biological disease that becomes lethal as it progresses with limited options for curing it beyond the early stage of localized cancer. Breast cancer, like many cancers, results from significant alterations in genetic and epigenetic mechanisms and targeting multiple signaling pathways in growth and malignant progression towards incurable lethal disease (Cai at al., Int. J. Mol. Sci. 2011, 12, 4465-4476). Recent results with multiple drug therapies have shown that cancer is a complex disease with tumor heterogeneity, rapid and dynamics of the tumor microenvironment that results in resistance to existing therapy are the most vexing challenging new treatment for breast cancer. Targeting a single cell-signaling pathway is unlikely to treat or prevent breast cancer. Combination therapy is a current strategy for breast cancer treatment and prevention (Zanardi et al., Semin. Oncol. 2015, 42, 887-895).

Curcumin (Natural yellow) is a phenolic compound extracted from the rhizome of Curcuma longa, the major ingredient in the spice, turmeric, and also in traditional medicines. Curcumin, a component of turmeric (Curcuma longa), is used as a remedy to treat a wide variety of ailments through a number of separate pharmacological pathways. Among the range of diseases curcumin is used to treat, it is more commonly used to treat inflammation without chronic side effects including gastrointestinal ulceration, kidney failure, and liver failure, and a considerable amount of research is currently being conducted to determine its anticancer, anti-inflammatory and antimicrobial capacity.

On the other hand, dichloroacetic acid (DCA), is a lead compound for treatment against BC since 2007 (Bonnet et al., Cancer Cell 2007, 11, 37-51). DCA is an inhibitor of pyruvate dehydrogenase kinase 1 (PDK1) of the pyruvate led glycolytic pathway in cancer cells because of its structural similarities with pyruvate, has recently attracted much attention as a potential anticancer drug for many human cancers including BC. DCA triggers in BC cells. DCA is very effective when used in combination with other drugs (Florio et al., Sci. Rep. 2018, 8, 13610; Khan et al., World J. Clin. Cases 2016, 4, 336-343; Alkarakooly et al., PLoS One 2018, 13, e0206182). However, a critical barrier in using DCA as an anticancer drug is that DCA inhibits PDK1 at micromolar concentration but much higher doses (˜100 times more) of this drug are needed for anticancer efficacy. Such high doses are frequently associated with neuropathy and other adverse side effects, which limits its therapeutic usefulness in cancer patients.

Current anti-inflammatory medications and cancer treatments, although effective, can produce serious side effects, which in some cases can be irreversible. For example, although common anti-inflammatory drugs such as analgesics and non-steroidal anti-inflammatory drugs (NSAIDs) such as ibuprofen, mefenamic acid, diclofenac, naproxen, and indomethacin, have been proven to manage pain and swelling, they are relatively inefficient and can produce significant side effects with prolonged use.

Although curcumin and DCA exhibit qualities that show promise for effectively treating certain life-threatening diseases, they do come with some drawbacks. Curcumin, while non-toxic, has low bioavailability and DCA can cause neurotoxicity in high concentrations. Eliminating these complications are important in developing possible curcumin and DCA drug treatments.

Therefore, there remains a need for improved compositions and methods of use thereof for the treatment of cancer.

It is another object of the invention to provide compositions with higher potency, greater bioavailability, fewer or decreased side effects, or a combination thereof and methods of using them for treating a wide range of cancers.

### SUMMARY OF THE INVENTION

Curcumin-based conjugates and methods of use thereof are provided. In one aspect, the invention provides curcumin conjugates having the general Formula I:

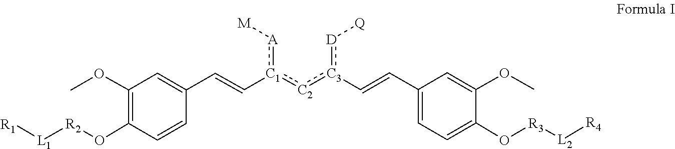

wherein the dotted lines between A and C₁, C₁ and C₂, C₂ and C₃, and C₃ and D indicate that a single or double bond may be present, as valence permits,

wherein the dotted lines A and M, and D and Q indicate that a single bond or no bond may be present, as valence permit,

wherein C₁, C₂, and C₃ are carbon atoms,

wherein A and D are oxygen atoms,

wherein M and Q are independently absent, or hydrogen, as valence permits,

wherein R₂ and R₃ can be independently absent, one or more amino acids or salts thereof, nucleic acids, lipids, polysaccharides, polymers, substituted or unsubstituted carbonyl groups, alkyl groups, alkenyl groups, alkynyl groups, aryl groups, heteroaryl groups, or other organic groups containing between C₁ and C₃₀ carbon atoms, inclusive, preferably between C₁ and C₂₀ carbon atoms, inclusive, more preferably between C₁ and C₁₀ carbon atoms, with the proviso that at least one of R₂ or R₃ is present,

wherein R₁ and R₄ can be independently absent, one or more amino acids or salts thereof, nucleic acids, lipids, polysaccharides, polymers, substituted or unsubstituted halogen groups, alkyl groups, alkenyl groups, alkynyl groups, aryl groups, heteroaryl groups, organic protecting groups, small molecules, or other organic groups containing between C₁ and C₃₀ carbon atoms, inclusive, preferably between C₁ and C₂₀ carbon atoms, inclusive, more preferably between C₁ and C₁₀ carbon atoms, and

wherein L₁ and L₂ can be independently absent, substituted or unsubstituted amide groups, halogen groups, alkyl groups, alkenyl groups, alkynyl groups, aryl groups, heteroaryl groups, or other organic groups containing between C₁ and C₂₀ carbon atoms, inclusive, preferably between C₁ and C₁₀ carbon atoms, inclusive, or pharmaceutically acceptable salt(s), polymorph(s), solvent(s), hydrate(s), crystal forms, and/or enantiomer(s) thereof.

One embodiment provides an optically pure composition containing one or more of the disclosed curcumin compositions. The optical purity is not particularly limited, but is usually about 90%, 95%, or 99% optically pure. Another embodiment provides a composition containing one or more of the disclosed curcumin compositions that is at least 99% optically pure.

In some aspects, the curcumin can be in the keto form, i.e., M and Q are absent, the bond between A and C₁, and D and C₃ are double bonds, and the bonds between C₁ and C₂, and C₂ and C₃ are single bonds.

In some aspects, the curcumin can be in the enol form, i.e., (i) the bond between C₁ and A is a double bond, M is absent, the bond between C₁ and C₂ is a single bond, the bond between C₂ and C₃ is a double bond, the bond between C₃ and D is a single bond, and Q is hydrogen, or (ii) the bond between C₃ and D is a double bond, Q is absent, the bond between C₂ and C₃ is a single bond, the bond between C₁ and C₂ is a double bond, the bond between C₁ and A is a single bond, and M is hydrogen.

Pharmaceutical compositions including an effective amount of one or more curcumin conjugates, for example, a mixture of two or more different curcumin conjugates, are also provided. The pharmaceutical compositions may include a pharmaceutically acceptable excipient. In particular embodiments, the compositions are formulated for enteral administration, for example oral administration. Other embodiments provide formulations for parenteral administration.

In a further aspect, the invention provides a method treating a cancer in a subject in need thereof comprising administering to the subject a therapeutically effective amount of the curcumin conjugate or a pharmaceutical composition thereof. In another aspect, the invention provides a method inhibiting cancer cell growth in a subject in need thereof comprising administering to the subject a therapeutically effective amount of the curcumin conjugate or a pharmaceutical composition thereof.

### BRIEF DESCRIPTION OF THE DRAWINGS

FIG. 1 shows DCA-curcumin hybrid conjugates, CMC 1-CMC 6, inhibit human breast cancer cell growth at nanomolar (nM) concentration.

FIG. 2 shows DCA-curcumin hybrid conjugates, CMC 1-CMC 6, inhibit colony formation in human breast cancer cells.

FIGS. 3A-3D shows CMC 2 treatment inhibits the tumor growth of genetically engineered mouse (GEM) model of breast cancer. Further, CMC2 treatment significantly reduced tumor growth (FIG. 3A), tumor size (FIG. 3B), and tumor weight (FIG. 3C).

FIGS. 4A-4E shows that CMC compounds are safe and do not show any contraindications.

### DETAILED DESCRIPTION OF THE INVENTION

### I. Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term ‘substituted’ is to be defined as set out below. It should be further understood that the terms “groups” and “radicals” can be considered interchangeable when used herein.

The articles “a” and “an” may be used herein to refer to one or to more than one (i.e., at least one) of the grammatical objects of the article. By way of example “an analogue” means one analogue or more than one analogue.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the term “pharmaceutically acceptable salts” includes acid addition salts or addition salts of free bases. “Pharmaceutically acceptable salts” of the disclosed compounds also include all the possible isomers and their mixtures, and any pharmaceutically acceptable metabolite, bioprecursor and/or pro-drug.

As used herein, the term “pro-drug” means a compound which has a structural formula different from a reference compound, and yet is directly or indirectly converted in vivo into the reference compound, upon administration to a subject, such as a mammal, particularly a human being.

The term, “alkyl,” as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycicoalkenyl, cycloalkynyl groups, alkyl substituted cycloalkyl, cycicoalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone, preferably 20 or fewer, and more preferably 10 or fewer.

The term, “alkyl,” also includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclic, aromatic or heteroaromatic moieties, —CF₃; —CN; —NCOCOCH₂CH₂; —NCOCOCHCH; —NCS; and combinations thereof.

The terms “alkenyl” and “alkynyl”, as used herein, refer to unsaturated aliphatic groups analogous in length and possible substitution to the alkyls described above, but that contain at least one double or triple bond respectively.

The term “aryl” refers to a mono- or multi-cyclic aromatic radical having in the range of 6 up to 30 carbon atoms such as phenyl, naphthyl, tetrahydronapthyl, indanyl, and biphenyl.

The term, “heteroaryl,” as used herein, refers to straight or branched chain, or cyclic carbon-containing radicals, or combinations thereof, having 3 to 30 carbon atoms where one or more of the carbon atoms are replaced by heteroatoms. Suitable heteroatoms include, but are not limited to, O, N, Si, P and S, where the nitrogen, phosphorous and sulfur atoms are optionally oxidized, and the nitrogen heteroatom is optionally quaternized. One of the rings may also be aromatic. Examples of heterocyclic and heteroaromatic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

The term “racemic” as used herein refers to a mixture of the (+) and (−) enantiomers of a compound wherein the (+) and (−) enantiomers are present in approximately a 1:1 ratio.

The terms “substantially optically pure,” “optically pure,” and “optically pure enantiomers,” as used herein, mean that the composition contains greater than about 90% of a single stereoisomer by weight, preferably greater than about 95% of the desired enantiomer by weight, and more preferably greater than about 99% of the desired enantiomer by weight, based upon the total weight.

### II. Compositions

A. Curcumin Conjugates

1. Structure of the Conjugates

Curcumin conjugates are provided. One embodiment provides curcumin conjugates having the following general Formula I:

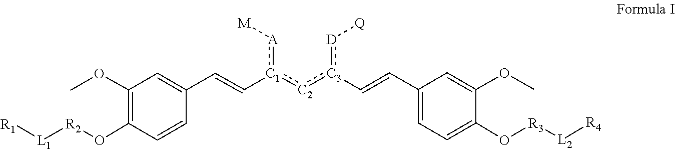

wherein the dotted lines between A and C₁, C₁ and C₂, C₂ and C₃, and C₃ and D indicate that a single or double bond may be present, as valence permits,

wherein the dotted lines A and M, and D and Q indicate that a single bond or no bond may be present, as valence permit,

wherein C₁, C₂, and C₃ are carbon atoms,

wherein A and D are oxygen atoms,

wherein M and Q are independently absent, or hydrogen, as valence permits,

wherein R₂ and R₃ can be independently absent, one or more amino acids or salts thereof, nucleic acids, lipids, polysaccharides, polymers, substituted or unsubstituted carbonyl groups, alkyl groups, alkenyl groups, alkynyl groups, aryl groups, heteroaryl groups, or other organic groups containing between C₁ and C₃₀ carbon atoms, inclusive, preferably between C₁ and C₂₀ carbon atoms, inclusive, more preferably between C₁ and C₁₀ carbon atoms, with the proviso that at least one of R₂ or R₃ is present,

wherein R₁ and R₄ can be independently absent, one or more amino acids or salts thereof, nucleic acids, lipids, polysaccharides, polymers, substituted or unsubstituted halogen groups, alkyl groups, alkenyl groups, alkynyl groups, aryl groups, heteroaryl groups, organic protecting groups, small molecules, or other organic groups containing between C₁ and C₃₀ carbon atoms, inclusive, preferably between C₁ and C₂₀ carbon atoms, inclusive, more preferably between C₁ and C₁₀ carbon atoms, and

wherein L₁ and L₂ can be independently absent, substituted or unsubstituted amide groups, halogen groups, alkyl groups, alkenyl groups, alkynyl groups, aryl groups, heteroaryl groups, or other organic groups containing between C₁ and C₂₀ carbon atoms, inclusive, preferably between C₁ and C₁₀ carbon atoms, inclusive, or pharmaceutically acceptable salt(s), polymorph(s), solvent(s), hydrate(s), crystal forms, and/or enantiomer(s) thereof.

In some embodiments, the curcumin is in the keto form, enol form or combinations thereof.

In other embodiment, R₁ and R₄ are each independently substituted or unsubstituted halogen groups.

In another embodiment, R₁ and R₄ are each independently dichloroacetic acid.

In certain embodiments, R₂ and R₃ are one or more amino acids or salts thereof.

In some embodiment, R₂ and R₃ are each independently substituted or unsubstituted carbonyl groups.

In other embodiment, L₁ and L₂ are each independently substituted or unsubstituted alkyl groups.

In another embodiment, L₁ and L₂ are each independently substituted or unsubstituted amide groups.

In some embodiments, the curcumin conjugate is selected from the group consisting of the structure of any one of the following compounds:

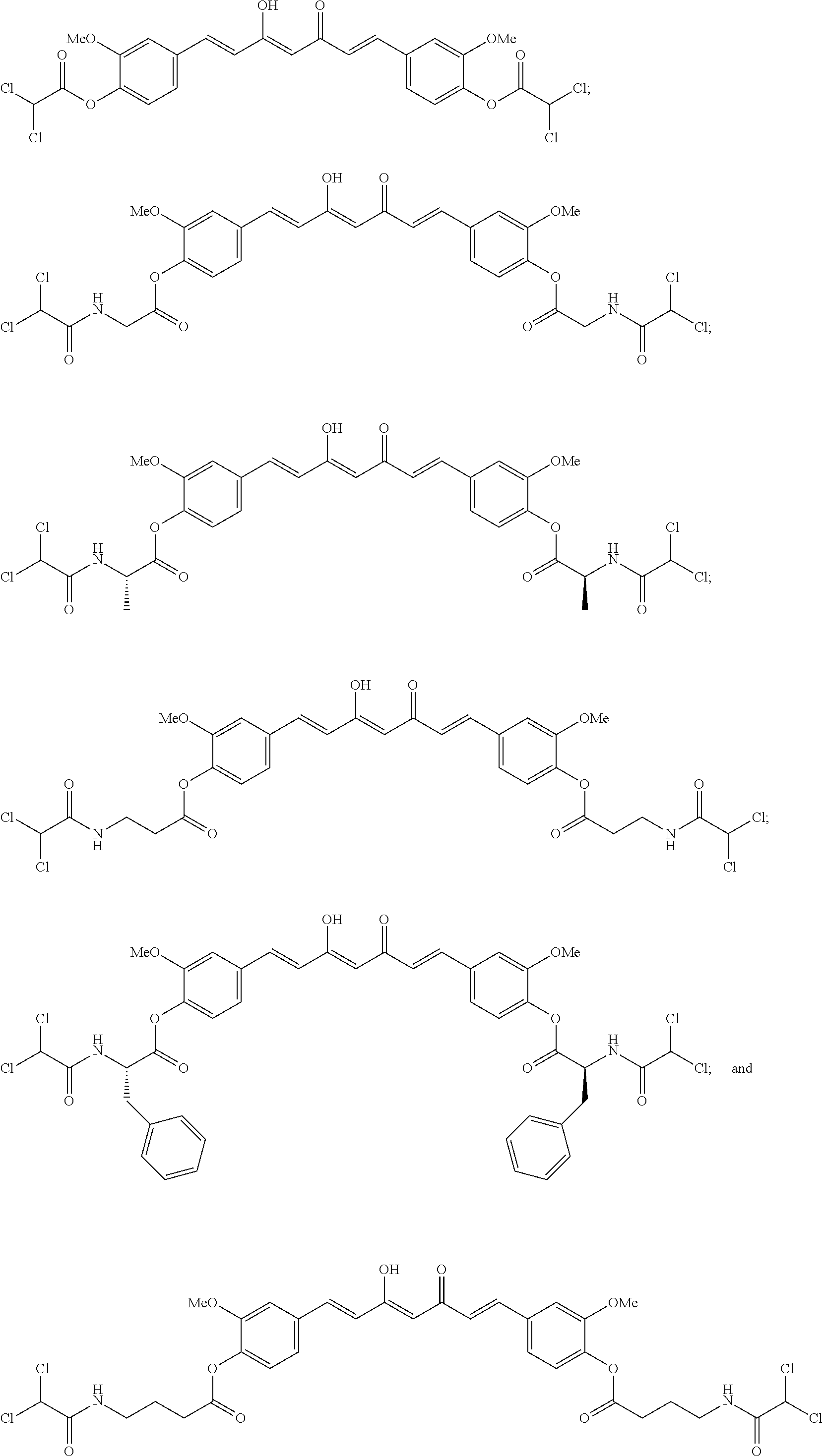

or pharmaceutically acceptable salt(s), polymorph(s), solvent(s), hydrate(s), crystal forms, and/or enantiomer(s) thereof.

One embodiment provides an optically pure composition containing one or more of the disclosed curcumin compositions. The optical purity is not particularly limited, but is usually about 90%, 95%, or 99% optically pure. Another embodiment provides a composition containing one or more of the disclosed curcumin compositions that is at least 99% optically pure.

The polymer can be, for example, a biodegradable polymer such as those known in the art. The polymer can be a hydrophobic polymer such as poly(ethylene glycol) (PEG), wherein the molecular weight is determined by the number of ethylene glycol units. For example, in some embodiments the PEG is between about 500 Da and 20,000 Da.

In some embodiments, the curcumin can be in the keto form, i.e., M and Q are absent, the bond between A and C₁ and D and C₃ are double bonds, and the bonds between C₁ and C₂ and C₂ and C₃ are single bonds.

In other embodiments, the curcumin can be in the enol form, i.e., (i) the bond between C₁ and A is a double bond, M is absent, the bond between C₁ and C₂ is a single bond, the bond between C₂ and C₃ is a double bond, the bond between C₃ and D is a single bond, and Q is hydrogen, or (ii) the bond between C₃ and D is a double bond, Q is absent, the bond between C₂ and C₃ is a single bond, the bond between C₁ and C₂ is a double bond, the bond between C₁ and A is a single bond, and M is hydrogen.

In some embodiments, the conjugates have one or more amino acids conjugated directly or indirectly thereto. The two or more amino acids can be the same or different amino acids. Thus, the curcumin conjugates disclosed herein can include the formula: AA1-C or C-AA1 or AA1-C-AA1 or AA2-C-AA1 or AA1-C-AA2, wherein “AA1” and “AA2” represent different amino acids, and “C” represents curcumin. In some embodiments, there is a linker or another molecule or moiety between curcumin and one or both amino acids. In a particularly preferred embodiment, the curcumin conjugate has the structure AA1-C-AA1.

As discussed in more detail below, one or both amino acids are typically conjugated to the curcumin or a linker linking it to curcumin by its C-terminal end. In other embodiments, one or both amino acids are conjugated to curcumin or a linker linking it to curcumin by its N-terminal end, its side group, or a combination thereof. In some embodiments, the end of the amino acid that is not conjugated to curcumin is free. In other embodiments, the end of the amino acid that is not conjugated to curcumin is conjugated to another moiety.

a. Curcumin Conjugates

One embodiment provides (1E,6E)-1,7-Bis(4-hydroxy-3-methoxyphenyl)hepta-1,6-diene-3,5-dione having the structure

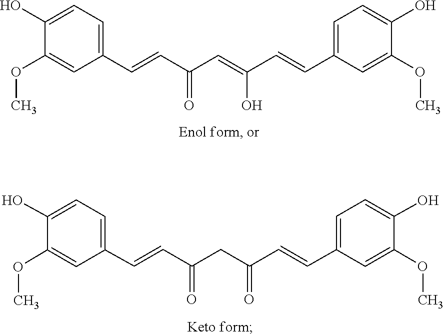 [SMILES: COc1cc(/C=C/C(=O)/C=C(O)/C=C/c2ccc(O)c(OC)c2)ccc1O ; COc1cc(/C=C/C(=O)CC(=O)/C=C/c2ccc(O)c(OC)c2)ccc1O]

or a variant, derivative, derivative, mimetic, prodrug, or mixtures thereof, or pharmaceutically acceptable salts thereof. The term “derivative” or “derivatized” as used herein includes one or more chemical modifications of (1E,6E)-1,7-Bis(4-hydroxy-3-methoxyphenyl)hepta-1,6-diene-3,5-dione. The term curcumin derivative means natural and synthetic curcumin derivatives. Examples include naturally occurring curcuminoids. These are plant secondary metabolites that occur in the rootstocks of different curcuma plants such as e.g. turmeric [curcuma Tonga]. The term curcuminoids covers the three substances curcumin, demethoxycurcumin and bisdemethoxycurcumin. From a chemical point of view, curcuminoids are conjugated diarylheptanoids, i.e., polyphenols in the broader sense. Curcumin derivatives are discussed in, for example, U.S. Published Application Nos. 2016/0213626, 2015/0342904, 2012/0316203, 2006/0276536, 2001/0051184 and U.S. Pat. Nos. 8,609,723, 8,956,589, 9,271,493, 9,446,145.

The disclosed compounds include compounds that are chemically modified to increase the resistance of the compound to enzymatic degradation, increase the half-life of the compound in vivo, reduce dosing frequency of the compound, decrease immunogenicity of the compound, increase the physical and/or thermal stability of the compound, increase the solubility of compound, increase the liquid stability of compound and/or reduce the aggregation of compound, and increase the purity of the active pharmaceutical ingredient in the final drug product. The addition of a soluble polymer or carbohydrate to compound may affect all of these pharmacokinetic parameters. The compound can also be one that has been chemically modified. Other forms of curcumin such as pharmaceutically acceptable salt(s), polymorph(s), solvent(s), hydrate(s), crystal forms, and/or enantiomer(s) may are also provided for use in the disclosed compositions and methods.

b. Amino Acids

As discussed above, some of the curcumin conjugates include one or more amino acids. The amino acid(s) can be a standard or non-standard amino acid. “Standard amino acid” or “canonical amino acid” typically refers to the twenty amino acids that are encoded directly by the codons of the universal genetic code denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Non-standard amino acid (nsAA)” refers to any and all amino acids that are not a standard amino acid. nsAA can be created by enzymes through posttranslational modifications; or those that are not found in nature and are entirely synthetic (e.g., synthetic amino acids (sAA)). In both classes, the nsAAs can be made synthetically. For example, in some embodiments, a tyrosine residue is substituted for a synthetic tyrosine derivative. WO 2015/120287 provides a non-exhaustive list of exemplary non-standard and synthetic amino acids that are known in the art (see, e.g., Table 11 of WO 2015/120287).

The amino acid(s) can be “D” amino acid(s), “L” amino acid(s), or a combination thereof. In some embodiments, the composition includes a mixture of curcumin conjugates. In some embodiments the mixture of curcumin conjugates includes one or more of the conjugates include one or more D amino acids and one or more of the conjugates include one or more L amino acids. In some embodiments, a curcumin conjugate includes at least one D amino acid and one L amino acid. The D and L amino acids can have the same or different side chains.

In some embodiments, the curcumin conjugates may include a salt form of the amino acid. That is, the one or more amino acids conjugated directly or indirectly to the disclosed curcumin conjugates may include a salt form of the amino acid. For example, the curcumin conjugates may include hydrochloride salt forms of the amino acid. In another embodiment, the curcumin conjugates may include acetate salt forms of the amino acid.

c. Additional Moieties and Linkers

In some embodiments, there is a linker or another molecule or moiety between curcumin and one or both amino acids; a linker or another molecule or moiety attached to the end of the amino acid that is not conjugated or linked to curcumin; or a combination thereof. Exemplary moieties include, but are not limited to nucleic acids and polynucleotides, amino acids and polypeptides, lipids, polysaccharides, small molecules, and protection groups.

In particular embodiments, the small molecule is a drug such as dichloroacetic acid (DCA):

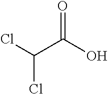

In some embodiments, the amino acid end group is protected. For example in some embodiments the conjugate has the formula Pg-AA1-C-AA1-Pg or Pg-AA2-C-AA1-Pg or Pg-AA1-C-AA2-Pg or C-AA1-Pg, or Pg-AA1-C wherein “AA1” and “AA2” represent different amino acids, and “C” represents curcumin, and “Pg” represents an amino-acid protecting group or diachloroacetic acid. Amino acid-protecting groups and method of use thereof are well known in the art. See, for example, Isidro-Llobet, et al., Chem. Rev., 109 (6):2455-2504 (2009), which is specifically incorporated by reference herein in its entirety. Suitable amine protecting groups include, but are not limited to, carbobenzyloxy (Cbz), p-Methoxybenzyl carbonyl (Moz or MeOZ), tert-butyloxycarbonyl (Boc), Fluorenylmethyloxycarbonyl (FMOC) carbamate, acetyl (Ac), benzoyl (Bz), benzyl (Bn), p-Methoxybenzyl (PMB), 3,4-Dimethoxybenzyl (DMPM), p-methoxyphenyl (PMP) group, tosyl (Ts), and trichloroethyl chloroformate (Troc). In particular embodiments, the protecting group is carboxybenzyl (Cbz), Fluorenylmethyloxycarbonyl (FMOC) carbamate, or tert-butyloxycarbonyl (Boc).

2. Exemplary Curcumin Conjugates

It is believed that coupling curcumin with amino acids and DCA, will yield potent hybrid molecules with greater than additive qualities and diminished side effects. To do this, optimal reaction conditions were established, which involved utilizing different coupling reagents and solvents at varied temperatures. Once favorable conditions were obtained, several curcumin-amino acid conjugates and curcumin-amino acid-DCA hybrid conjugates were successfully synthesized in excellent yield without alterations to chirality. In doing so, an efficient methodology for synthesizing these conjugates was developed. Exemplary curcumin conjugates are illustrated in Table 1 below.

a. Curcumin-DCA Hybrids

In some embodiments, the curcumin conjugate is a curcumin-DCA hybrid conjugate that includes or is any one of compounds 3-1 to 3-13 of Table 1.

TABLE 1
DCA-Amino Acid-Curcumin Hybrid Conjugates

| S. No | Structure | IUPAC name |
|---|---|---|
| 3-1 | 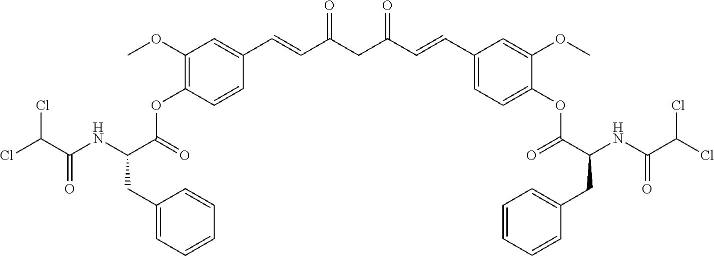 [SMILES: COc1cc(/C=C/C(=O)CC(=O)/C=C/c2ccc(OC(=O)[C@H](Cc3ccccc3)NC(=O)C(Cl)Cl)c(OC)c2)ccc1OC(=O)[C@H](Cc1ccccc1)NC(=O)C(Cl)Cl] | ((1E,6E)-3,5- dioxohepta-1,6- diene-1,7-diyl) bis(2-methoxy- 4,1-phenylene) (2S,2′S)-bis(2- (2,2- dichloroacetamido)-3- phenylpropanoate) |
| 3-2 | 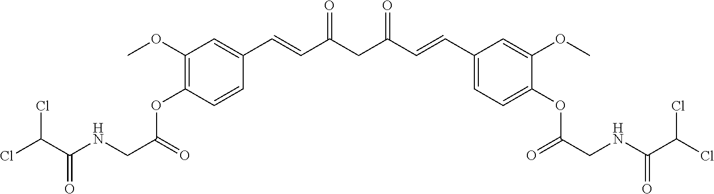 [SMILES: COc1cc(/C=C/C(=O)CC(=O)/C=C/c2ccc(OC(=O)CNC(=O)C(Cl)Cl)c(OC)c2)ccc1OC(=O)CNC(=O)C(Cl)Cl] | ((1E,6E)-3,5- dioxohepta-1,6- diene-1,7-diyl) bis(2-methoxy- 4,1-phenylene) bis(2-(2,2- dichloroacetamido) acetate) |
| 3-3 | 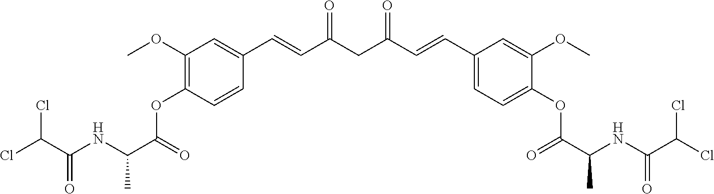 [SMILES: COc1cc(/C=C/C(=O)CC(=O)/C=C/c2ccc(OC(=O)[C@H](C)NC(=O)C(Cl)Cl)c(OC)c2)ccc1OC(=O)[C@H](C)NC(=O)C(Cl)Cl] | ((1E,6E)-3,5- dioxohepta-1,6- diene-1,7-diyl) bis(2-methoxy- 4,1-phenylene) (2S,2′S)-bis(2- (2,2- dichloroacetamido) propanoate) |
| 3-4 | 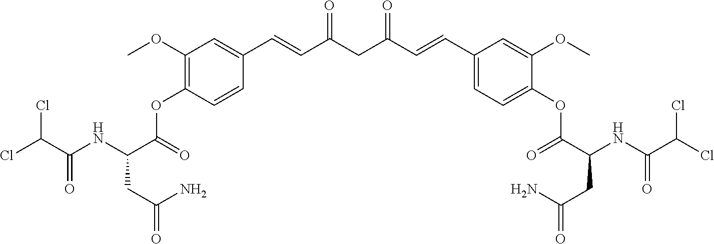 [SMILES: COc1cc(/C=C/C(=O)CC(=O)/C=C/c2ccc(OC(=O)[C@H](CC(N)=O)NC(=O)C(Cl)Cl)c(OC)c2)ccc1OC(=O)[C@H](CC(N)=O)NC(=O)C(Cl)Cl] | ((1E,6E)-3,5- dioxohepta-1,6- diene-1,7-diyl) bis(2-methoxy- 4,1-phenylene) (2S,2′S)-bis(4- amino-2-(2,2- dichloroacetamido)-4- oxobutanoate) |
| 3-5 | 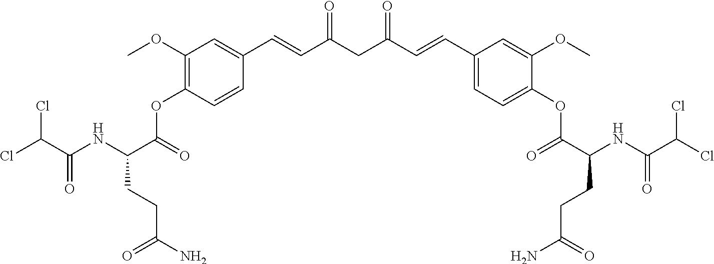 [SMILES: COc1cc(/C=C/C(=O)CC(=O)/C=C/c2ccc(OC(=O)[C@H](CCC(N)=O)NC(=O)C(Cl)Cl)c(OC)c2)ccc1OC(=O)[C@H](CCC(N)=O)NC(=O)C(Cl)Cl] | ((1E,6E)-3,5- dioxohepta-1,6- diene-1,7-diyl) bis(2-methoxy- 4,1-phenylene) (2S,2′S)-bis(5- amino-2-(2,2- dichloroacetamido)-5- oxopentanoate) |
| 3-6 | 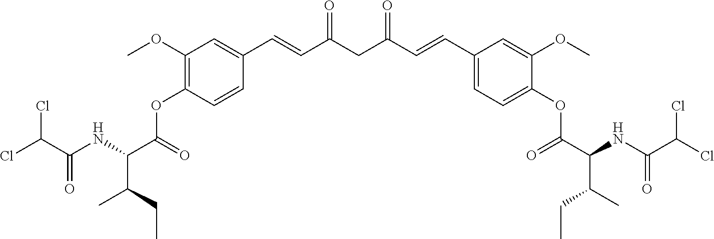 [SMILES: CC[C@@H](C)[C@H](NC(=O)C(Cl)Cl)C(=O)Oc1ccc(/C=C/C(=O)CC(=O)/C=C/c2ccc(OC(=O)[C@@H](NC(=O)C(Cl)Cl)[C@H](C)CC)c(OC)c2)cc1OC] | ((1E,6E)-3,5- dioxohepta-1,6- diene-1,7-diyl) bis(2-methoxy- 4,1-phenylene) (2S,2′S,3R,3′R)- bis(2-(2,2- dichloroacetamido)-3- methylpentanoate) |
| 3-7 | 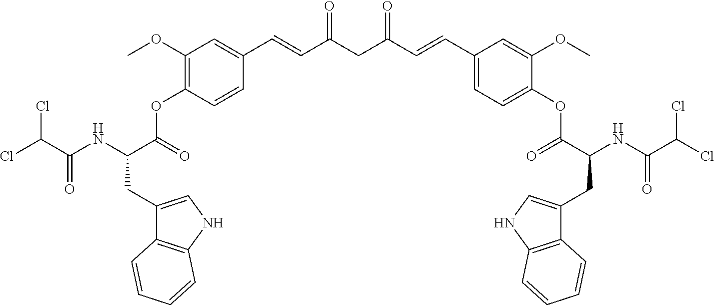 [SMILES: COc1cc(/C=C/C(=O)CC(=O)/C=C/c2ccc(OC(=O)[C@H](Cc3c[nH]c4ccccc34)NC(=O)C(Cl)Cl)c(OC)c2)ccc1OC(=O)[C@H](Cc1c[nH]c2ccccc12)NC(=O)C(Cl)Cl] | ((1E,6E)-3,5- dioxohepta-1,6- diene-1,7-diyl) bis(2-methoxy- 4,1-phenylene) (2S,2′S)-bis(2- (2,2- dichloroacetamido)-3- (1H-indol-3- yl)propanoate) |
| 3-8 | 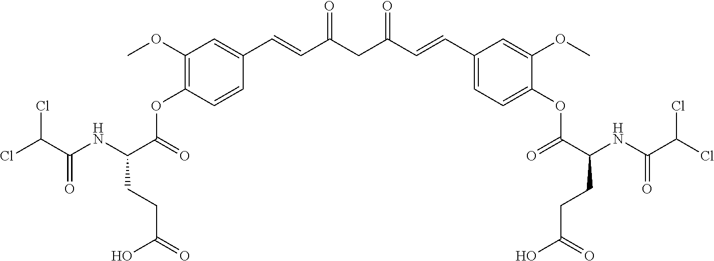 [SMILES: COc1cc(/C=C/C(=O)CC(=O)/C=C/c2ccc(OC(=O)[C@H](CCC(=O)O)NC(=O)C(Cl)Cl)c(OC)c2)ccc1OC(=O)[C@H](CCC(=O)O)NC(=O)C(Cl)Cl] | (4S,4′S)-5,5′- ((((1E,6E)-3,5- dioxohepta- 1,6-diene-1,7- diyl)bis(2- methoxy-4,1- phenylene)) bis(oxy))bis(4- (2,2- dichloroacetamido)-5- oxopentanoic acid) |
| 3-9 | 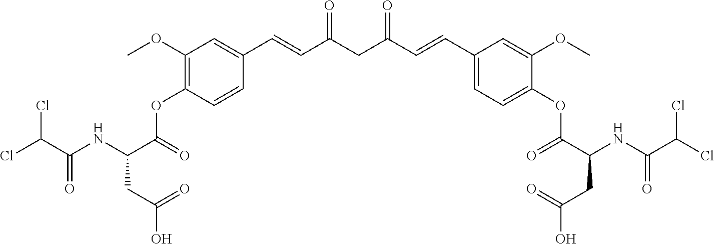 [SMILES: COc1cc(/C=C/C(=O)CC(=O)/C=C/c2ccc(OC(=O)[C@H](CC(=O)O)NC(=O)C(Cl)Cl)c(OC)c2)ccc1OC(=O)[C@H](CC(=O)O)NC(=O)C(Cl)Cl] | (3S,3′S)-4,4′- ((((1E,6E)-3,5- dioxohepta- 1,6-diene-1,7- diyl)bis(2- methoxy-4,1- phenylene)) bis(oxy))bis(3- (2,2- dichloroacetamido)-4- oxobutanoic acid) |
| 3-10 | 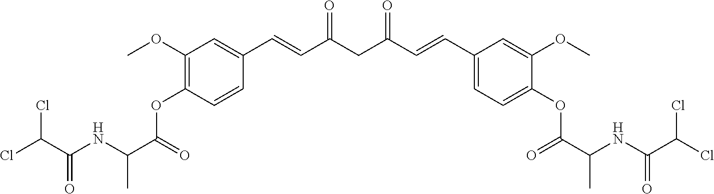 [SMILES: COc1cc(/C=C/C(=O)CC(=O)/C=C/c2ccc(OC(=O)C(C)NC(=O)C(Cl)Cl)c(OC)c2)ccc1OC(=O)C(C)NC(=O)C(Cl)Cl] | ((1E,6E)-3,5- dioxohepta-1,6- diene-1,7-diyl) bis(2-methoxy- 4,1-phenylene) bis(2-(2,2- dichloroacetamido) propanoate) |
| 3-11 | 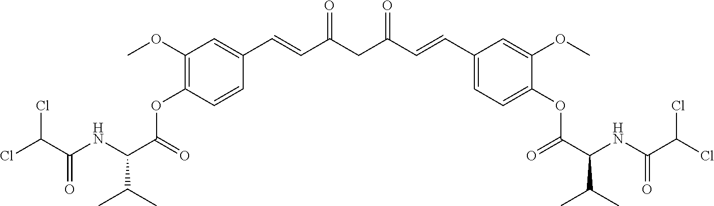 [SMILES: COc1cc(/C=C/C(=O)CC(=O)/C=C/c2ccc(OC(=O)[C@@H](NC(=O)C(Cl)Cl)C(C)C)c(OC)c2)ccc1OC(=O)[C@@H](NC(=O)C(Cl)Cl)C(C)C] | ((1E,6E)-3,5- dioxohepta-1,6- diene-1,7-diyl) bis(2-methoxy- 4,1-phenylene) (2S,2′S)-bis(2- (2,2- dichloroacetamido)-3- methylbutanoate) |
| 3-12 | 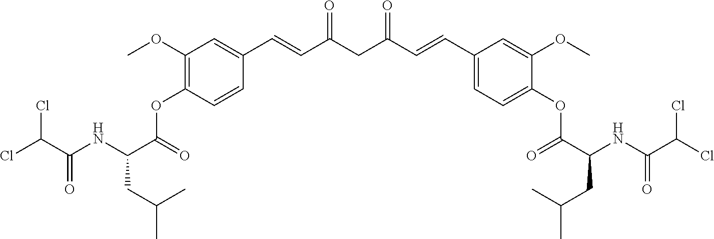 [SMILES: COc1cc(/C=C/C(=O)CC(=O)/C=C/c2ccc(OC(=O)[C@H](CC(C)C)NC(=O)C(Cl)Cl)c(OC)c2)ccc1OC(=O)[C@H](CC(C)C)NC(=O)C(Cl)Cl] | ((1E,6E)-3,5- dioxohepta-1,6- diene-1,7-diyl) bis(2-methoxy- 4,1-phenylene) (2S,2′S)-bis(2- (2,2- dichloroacetamido)-4- methylpentanoate) |
| 3-13 | 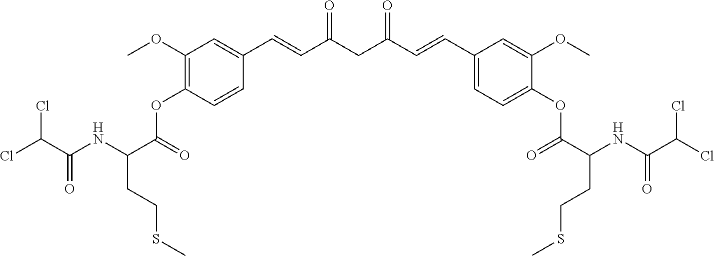 [SMILES: COc1cc(/C=C/C(=O)CC(=O)/C=C/c2ccc(OC(=O)C(CCSC)NC(=O)C(Cl)Cl)c(OC)c2)ccc1OC(=O)C(CCSC)NC(=O)C(Cl)Cl] | ((1E,6E)-3,5- dioxohepta-1,6- diene-1,7-diyl) bis(2-methoxy- 4,1-phenylene) bis(2-(2,2- dichloroacetamido)- 4- (methylthio) butanoate) |

B. Formulations and Pharmaceutical Compositions

In some embodiments the disclosed cucurmin conjugates and combinations thereof can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration. The compositions can be administered systemically.

In some embodiments, the disclosed pharmaceutical compositions containing the disclosed curcumin conjugates can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

The disclosed formulations can be prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et. al., (Media, Pa.: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

1. Formulations for Parenteral Administration

The disclosed curcumin conjugates and pharmaceutical compositions thereof can be administered in an aqueous solution, by parenteral injection. The formulation may also be in the form of a suspension or emulsion. In general, pharmaceutical compositions are provided including effective amounts of the disclosed curcumin conjucates and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Oral Immediate Release Formulations

One embodiment provides suitable oral dosage forms of the curcumin conjugates that include but are not limited to tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

In some embodiments binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

In some embodiments lubricants are used to facilitate tablet manufacture. Examples of suitable lubricants include, but are not limited to, magnesium stearate, calcium stearate, stearic acid, glycerol behenate, polyethylene glycol, talc, and mineral oil.

In some embodimetns disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

In some embodiments stabilizers are used to inhibit or retard drug decomposition reactions which include, by way of example, oxidative reactions.

In some embodiments surfactants are used and may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

3. Extended Release Dosage Forms

Some embodiments provide extended release formulations containing the disclosed curcumin conjugates that are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, the disclosed extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Some embodiments provide extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

4. Delayed Release Dosage Forms

Some embodiments provide delayed release formulations containing the disclosed curcumin conjugates that are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUIDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUIDRAGIT®. S (soluble at pH 7.0 and above, as a result of a higher degree of esterification), and EUIDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Methods of Manufacturing

As will be appreciated by those skilled in the art and as described in the pertinent texts and literature, a number of methods are available for preparing the disclosed curcumin conjugates containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

5. Formulations for Mucosal and Pulmonary Administration

Some embodiments provide the disclosed curcumin conjugates and compositions thereof formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the curcumin conjugates are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, the pharmaceutical compositions containing the disclosed curcumin conjugates may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PART LC Jet+ nebulizer (PART Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

6. Topical and Transdermal Formulations

Some embodiments provide transdermal formulations containing the disclosed curcumin conjugates. These will typically be gels, ointments, lotions, sprays, or patches, all of which can be prepared using standard technology. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

In some embodiments, the composition is formulated for transdermal delivery and administered using a transdermal patch. In some embodiments, the formulation, the patch, or both are designed for extended release of the curcumin conjugate.

Exemplary symptoms, pharmacologic, and physiologic effects are discussed in more detail below.

### III. Methods of Treatment

In one embodiment, one or more of the disclosed curcumin conjugates a curcumin conjugate can be administered to a subject in need thereof in an effective amount to treat a disease or disorder or otherwise provide a desired pharmacologic and/or physiologic effect. In one embodiment, the disease is cancer, including but not limited to breast cancer.

In some embodiments, the curcumin conjugates are used to treat a disease or disorder or induce or increase a physiological effect previously identified as treatable by curcumin. For example, curcumin regulates the expression of inflammatory enzymes, cytokines, adhesion molecules, and cell survival proteins by modulating the activation of various transcription factors (Goel, et al., Biochemical Pharmacology, 75(4):787-809 (2008).

Curcumin also downregulates cyclin D1, cyclin E and MDM2; and upregulates p21, p2′7, and p53, and various preclinical cell culture and animal studies indicate that curcumin can be used as an antiproliferative, anti-invasive, and antiangiogenic agent; as a mediator of chemoresistance and radioresistance; as a chemopreventive agent; and as a therapeutic agent in wound healing, diabetes, neurodegenerative diseases such as Alzheimer disease and Parkinson disease, cardiovascular disease, pulmonary disease, and arthritis (Goel, et al., Biochemical Pharmacology, 75(4):787-809 (2008). Clinical trials clinical trial supports a therapeutic role for curcumin in diseases such as familial adenomatous polyposis, inflammatory bowel disease, ulcerative colitis, colon cancer, pancreatic cancer, hypercholesteremia, atherosclerosis, pancreatitis, psoriasis, chronic anterior uveitis and arthritis. As discussed in more detail below, in some embodiments, the conjugates are used to treat inflammation, cancer, or an infection.

In some embodiments, the effect of the composition on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects).

In some embodiments, the effect of the treatment is compared to a conventional treatment that is known the art, such as one of those discussed herein. Preferably, the disclosed compositions have less toxicity than curcumin at the same dosage, a greater potency or other pharmacological effect than curcumin at the same dosage, or a combination thereof. In some embodiments, the compositions can be administered at a lower dosage than curcumin, but achieve a greater therapeutic effect, lower toxicity, or a combination thereof.

Pilot phase I clinical trials have shown curcumin to be safe even when consumed at a daily dose of 12 g for 3 months (Goel, et al., Biochemical Pharmacology, 75(4):787-809 (2008)). In general, by way of example only, dosage forms useful in the disclosed methods can include doses in the range of 0.1 mg to 25 g, 100 mg to 20 g, 100 mg to 15 g, with doses of 1 mg, 5 mg, 7.5 mg, 10 mg, 25 mg, 50 mg, 75 mg, 100 mg, 250 mg, 500 mg, 750 mg, 1 g, 2.5 g, 5 g, 7.5 g, and 10 g being specific examples of doses. Typically, such dosages are administered once, twice, or three times daily, or once every 1, 2, 3, 4, 5, 6, or 7 days' day to a human.

Treatment of Cancer

Methods for preventing, treating, and/or managing cancer, can include administering to a subject in need thereof an effective amount of the disclosed curcumin conjugates. In other embodiments, methods for inhibiting cancer cell growth, can include administering to a subject in need thereof an effective amount of the disclosed curcumin conjugates. In a specific embodiment, the curcumin conjugate or a composition thereof is the only active agent administered to a subject (i.e., monotherapy) relative to a control. In certain embodiments, the subject is selected from the group consisting of mammal, human or genetically engineered mouse (GEM).

In some embodiments, the composition containing the disclosed curcumin conjugates increases cancer cell death, reduces tumor size, reduces cancer cell proliferation, reduce tumor growth, reduces tumor burden or a combination thereof relative to a control.

In some embodiments, the composition containing the disclosed curcumin conjugates achieves at least one, two, three, four or more of the following effects: (i) the reduction or amelioration of the severity of one or more symptoms of cancer; (ii) the reduction in the duration of one or more symptoms associated with cancer, for example breast cancer; (iii) the prevention in the recurrence of a symptom associated with cancer; (iv) the reduction in hospitalization of a subject; (v) a reduction in hospitalization length; (vi) the increase in the survival of a subject; (vii) the enhancement or improvement of the therapeutic effect of another therapy; (viii) an increase in the survival rate of patients; (xiii) a decrease in hospitalization rate; (ix) the prevention of the development or onset of one or more symptoms associated with cancer; (x) the reduction in the number of symptoms associated with cancer; (xi) an increase in symptom-free survival of cancer patients; (xii) improvement in quality of life as assessed by methods well known in the art; (xiii) the prevention in the recurrence of a tumor; (xiv) the regression of tumors and/or one or more symptoms associated therewith; (xvii) the inhibition of the progression of tumors and/or one or more symptoms associated therewith; (xviii) a reduction in the growth of a tumor; (xix) a decrease in tumor size (e.g., volume or diameter); (xx) a reduction in the formation of a newly formed tumor; (xxi) eradication, removal, or control of primary, regional and/or metastatic tumors; (xxii) a decrease in the number or size of metastases; (xxiii) a reduction in mortality; (xxiv) an increase in the tumor-free survival rate of patients; (xxv) an increase in relapse free survival; (xxvi) an increase in the number of patients in remission; (xxvii) the size of the tumor is maintained and does not increase or increases by less than the increase of a tumor after administration of a standard therapy as measured by conventional methods available to one of skill in the art, such as magnetic resonance imaging (MM), dynamic contrast-enhanced MM (DCE-MRI), X-ray, and computed tomography (CT) scan, or a positron emission tomography (PET) scan; and/or (xxviii) an increase in the length of remission in patients.

Cancers and related disorders that can be prevented, treated, or managed in accordance with the methods described herein include, but are not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, and chronic lymphocytic leukemia, hairy cell leukemia; polycythemia vera; lymphomas such as but not limited to Hodgkin's disease, and non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma; gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, semi noma, anaplastic, spermatocytic, nonseminoma, embryonal carcinoma, teratoma carcinoma, choriocarcinoma (yolk-sac tumor); prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, and superficial spreading melanoma, nodular melanoma, lentigo malignant melanoma, acral lentiginous melanoma; kidney cancers including but not limited to, renal cell cancer, renal cancer, adenocarcinoma, hypernephroma, fibrosarcoma, and transitional cell cancer (renal pelvis and/or uterer); Wilms' tumor; bladder cancers including but not limited to, transitional cell carcinoma, squamous cell cancer, adenocarcinoma, and carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In one embodiment, the cancer is benign, e.g., polyps and benign lesions. In other embodiments, the cancer is metastatic. The compositions can be used in the treatment of pre-malignant as well as malignant conditions. Pre-malignant conditions include hyperplasia, metaplasia, and dysplasia. Treatment of malignant conditions includes the treatment of primary as well as metastatic tumors. In a specific embodiment the cancer is melanoma, colon cancer, lung cancer, breast cancer, prostate cancer, cervical cancer, liver cancer, testicular cancer, brain cancer, pancreatic cancer, or renal cancer.

### IV. Combination Therapies

In some embodiments, the curcumin conjugate(s) is administered in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of curcumin conjugate(s). The different active agents can have the same or different mechanisms of action. In some embodiments, the combination results in an additive effect on the treatment of the disease or disorder. In some embodiments, the combinations result in a more than additive effect on the treatment of the disease or disorder. The additional active ingredients can be chemotherapeutic agents, immunomodulatory agents, and anti-inflammatory agents. For example, the disclosed compositions can be administered to a subject in need thereof in combination with: an antimicrobial such as an antibiotic, or an antifungal, or an antiviral, or an antiparasitic, or an essential oil, or a combination thereof.

Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof. The pharmaceutical compositions can be formulated as a pharmaceutical dosage unit, also referred to as a unit dosage form.

In particular embodiments, a combination therapy includes curcumin conjugate(s) and one or more conventional treatments for the disease or disorder to be treated, such as those discussed herein.

### EXAMPLES

### Example 1: Synthesis of DCA-Curcumin Conjugate CMC 1

Methods and Materials

The hybrid conjugate of DCA and curcumin was synthesized by treating DCA with curcumin in presence of N-(3-Dimethylaminopropyl)-N′-ethylcarbodiimide hydrochloride (EDAC) and DMAP (4-(Dimethylamino)pyridine) in DCM at −5° C. The isolated hybrid conjugate was further recrystallized with ethanol (Scheme 1).

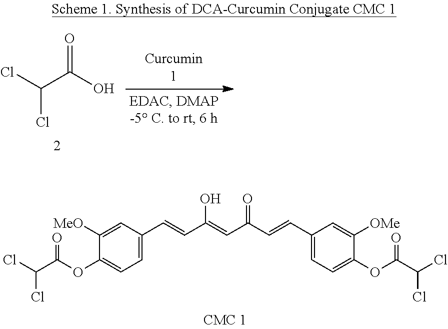 [SMILES: O=C(O)C(Cl)Cl ; COc1cc(/C=C/C(=O)/C=C(O)/C=C/c2ccc(OC(=O)C(Cl)Cl)c(OC)c2)ccc1OC(=O)C(Cl)Cl]

Results

An efficient methodology for synthesizing DCA-Curcumin Conjugate CMC 1 was developed. The synthesized CMC 1 compound was fully characterized by NMR and Mass spectroscopy. The purity and chiral integrity of CMC 1 compound was confirmed by optical rotation, chiral HPLC studies.

### Example 2: General Procedure for Synthesis of DCA-Curcumin Hybrid Conjugates CMC 2-CMC 6

Methods and Materials

DCA was activated by benzotriazole 3 by using our previously reported method [31]. The benzotriazole activated DCA 4 reacted with amino acids in the presence of TEA in aqueous acetonitrile at room temperature to form the DCA-amino acid conjugates 5a-e. Conjugates 5a-e further coupled with curcumin 1 with 2 to 1 ration under optimized reaction condition to obtain the hybrid conjugates CMC2-6 in good yields (Scheme 2).

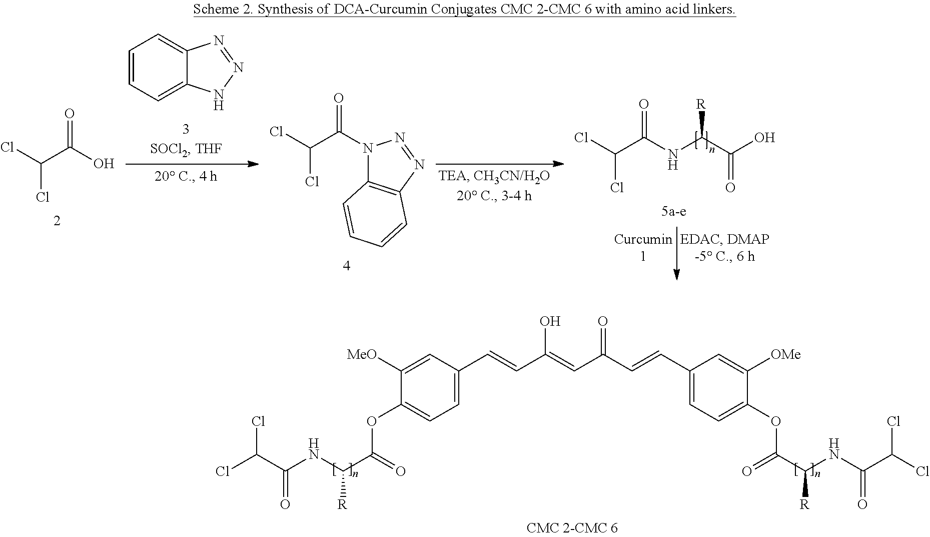

A dried round bottom flask containing a small stir bar was charged with curcumin (1.0 equiv.) and DCA or the respective protected DCA-amino acid (2.0 equiv.) dissolved in DCM (5 mL) along with EDAC (2.5 equiv.) and DMAP (0.5 equiv.). The reaction mixture was cooled down to −5° C. in an ice bath and continued stirring for 4-6 hours. The progress of each mixture was monitored through thin layered chromatography (TLC) and upon completion, the DCM was evaporated under reduced pressure. The residues were treated with 2N HCl (10 mL) and the solid obtained was filtered and washed with water (50 mL) to give the desired hybrid conjugates. We recrystallized the products with aqueous ethanol to get in pure form.

Results

An efficient methodology for synthesizing DCA-Curcumin Conjugates CMC 2-CMC 6 was developed. All the synthesized compounds were fully characterized by NMR and Mass spectroscopy. The purity and chiral integrity of the compounds were confirmed by optical rotation, chiral HPLC studies.

### Example 3: DCA-Curcumin Hybrid Conjugates Inhibit Human Breast Cancer Cell Growth at Nanomolar (nM) Concentration

Methods and Materials

We tested the antitumor potential of All the synthesized six DCA-curcumin hybrid conjugates (CMC 1-CMC 6) with two different human breast cancer cell lines [T47D, an ER-positive BC cell line and MDA-MB231 (MB231), a triple-receptor negative BC (TNBC) cell line] using the colorimetric MTT (3-(4,5-dimethylthiazol-2yl)-2,5-diphenyltetrazolium bromide) cell proliferation assay kit.

Results

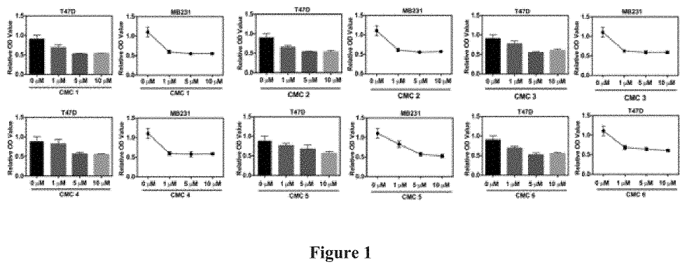

Most of these conjugates effectively inhibit cell proliferation at a nanomolar concentration (FIG. 1). Based on these results, we have calculated ECso values for these compounds and found that most of these CMC compounds inhibit BC cell growth at nanomolar to submicromolar concentrations (Table 2). These observations provide a strong rationale to test the hypothesis that synthesized conjugates have potential antitumor potential and it is imperative to establish the antitumor potential of these compounds in breast cancer.

FIG. 1 shows DCA-curcumin hybrid conjugates inhibit human breast cancer cell growth at nanomolar (nM) concentration. MCF7 and MB231 cells (5×10³) were seed in 96-well plates and left them in the cell culture incubator at 37° C. with 5% CO₂ in DMEM and RPMI medium (100 μl), respectively. After 24 h, the medium was replaced with the CMC compounds at different concentrations (0, 1, 5, and 10 μM) for 72 h. After 72 h, 10 μl Reliablue reagent was added to each well and incubated for 2 h for the formation of purple formazan and then added 100 μl of detergent to dissociate the formazan precipitate and measured at 570 nm. Values are shown as mean±SEM of three experiments with 6 wells in each, a total of 18 repeats.

TABLE 2
EC₅₀ values for CMC compounds (CMC 1-CMC 6) in two different
human breast cancer cell lines:

| Name of the | | |
|---|---|---|
| compound | EC₅₀ (nM) for T47D cells | EC₅₀ (nM) for MB231 cells |
| CMC 1 | 1.648 μM | 0.4240 μM |
| CMC 2 | 1.421 μM | 0.7780 μM |
| CMC 3 | 1.595 μM | 0.5179 μM |
| CMC 4 | 1.255 μM | 1.1320 μM |
| CMC 5 | 1.245 μM | 0.8375 μM |
| CMC 6 | 1.372 μM | 0.9418 μM |

### Example 4: DCA-Curcumin Hybrid Conjugates Inhibit Colony Formation in Human Breast Cancer Cells

Methods and Materials

To provide more evidence for the antitumor potential of these compounds in BC, we also analyzed the colony formation assay in two different human BC cell lines (T47D and MB231). For this assay, we platted T47 and MB231 cells (1×10³) in 24-well plates and cultured them in the incubator at 37° C. with 5% CO₂ in DMEM and RPMI medium (1.0 ml), respectively.

Results

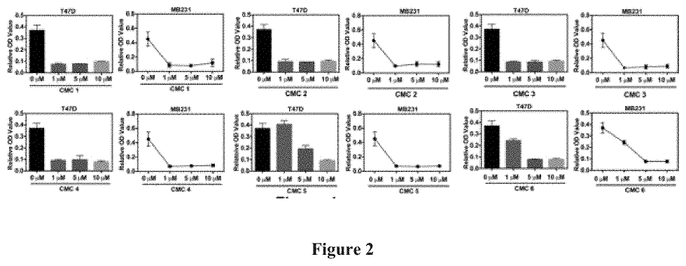

The colony formation assay also confirms the activity of the CMC compounds against T47D and MB231 cell lines. As shown in FIG. 2, most of the CMC compounds have significantly reduced colony formation at nanomolar to submicromolar concentrations.

FIG. 2 shows DCA-curcumin hybrid conjugates inhibit colony formation in human breast cancer cells. T47D and MB231 cells (1×10³) were seed in 24-well plates and left them in the cell culture incubator at 37° C. with 5% CO₂ in DMEM and RPMI medium (1.0 ml), respectively. After 24 h, cells were exposed to different CMC compounds at different concentrations (0, 1, 5, and 10 μM) for 2 weeks, changing the medium for every 3 days with respective CMC compounds at the indicated concentrations. After 2 weeks, cells were washed with PBS and fixed in 100% methanol for 30 minutes followed by staining with KaryoMax Giemsa stain for 1 h. The unfound dyes were removed by washing the wells with water and dried overnight at room temperature. Finally, the cells were lysed with lysis buffer (1% SDS in 0.2 N NaOH for 5 min, and the absorbance of the released dye was measured at 630 nm. Values are shown as mean±SEM of three experiments with 3 wells in each, a total of 9 repeats.

### Example 5: CMC 2 Treatment Inhibits the Tumor Growth Genetically Engineered Mouse (GEM) Model of Breast Cancer

Methods and Materials

The antitumor potential of one of the most potential CMC compound, CMC2, in genetically engineered mouse (GEM) model of breast cancer, MMTV-PyMT-Tg mice was tested. The MMTV-PyMT-Tg mouse was mainly chosen because the tumor formation and progression in this mouse is characterized by four different stages (hyperplasia, adenoma/mammary intra-epithelial neoplasia, early and late carcinoma) and also mimics human breast cancer, the tumor develops first as ER-positive (ER⁺) but ultimately becomes ER-negative BC (ER⁻ BC).

It was randomly assigned 6-week-old MMTV-PyMT-Tg mice into two groups (3 mice in each); one control and one CMC2 treated (10 mg/kg body, three times a week by oral gavage for 7 weeks). Tumor volume was measured twice a week and the volume was calculated using the formula V=L×W²/2, where L represents the largest tumor diameter and W represents the smallest tumor diameter. After the experimental period, tumor tissues were collected, a picture was taken, and tumor weight was measured. Tissue sections were prepared from the tumor tissue and stained for Hematoxylin and Eosin (H & E) section and also stained for the Ki67 for cell proliferation. Tissue sections and H & E sections were prepared from the Augusta University Histology core facility.

Results

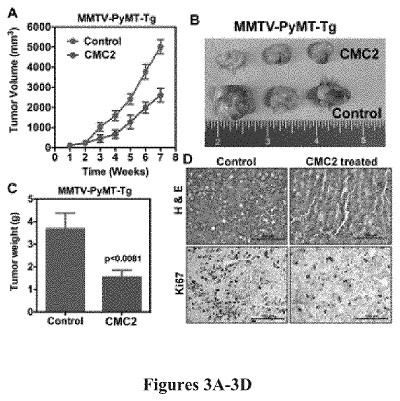

As shown in FIG. 3, CMC2 treatment significantly reduced tumor growth (FIG. 3A), tumor size (FIG. 3B), and tumor weight (FIG. 3C).

FIG. 3 shows CMC 2 treatment inhibits the tumor growth of genetically engineered mouse (GEM) model of breast cancer. Six-week-old MMTV-PyMT-Tg mice were treated with CMC2 (10 mg/kg body for 3× a week for 7 weeks. Control mice received PBS. Tumor volume was measured twice a week and calculated (A). After the experimental period, tumor size (B) and tumor weight (C) were also analyzed. Tissue sections were prepared and stained with H & E and Ki67 (D). Values are shown as mean±SD of three mice in each.

### Example 6: Adverse Effects and Toxicity—CMC Compounds are Safe and do not Show any Contraindication

Methods and Materials

Adverse effects and toxicity to the normal cells are the most challenging part of the anticancer drug development process. CMC1 and CMC 2 were administrated to the mouse via oral gavage (50 mg/kg body and 100 mg/kg body) for 7 days. The body weight and any side effects of contraindication monitored during the period.

Results

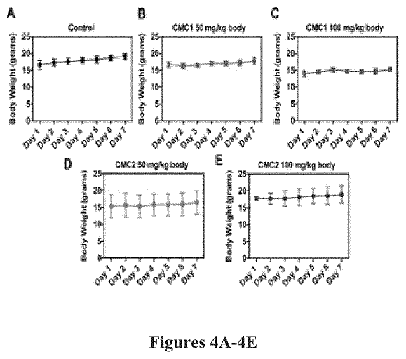

As shown in FIG. 4A-FIG. 4E, the mouse growth was normal and there was no contraindication suggesting that the CMC1 and CMC2 are very safe and can be used for the preclinical analysis.

FIG. 4 shows that CMC compounds are safe and do not show any contraindication. Six-week-old normal control FVB/N mice were treated with CMC1 and CMC 2 (50 and 100 mg/kg body for 3× a week for oral gavage). Control mice received PBS. After one weak of drug administration, mouse weight was measured every day for 7 days. We used 3 mice in each group. (A) Body weight changes in control mice (B and C) Body weight changes in CMC1 (50 and 100 mg/kg body). (D and E) Body weight changes in CMC1 (50 and 100 mg/kg body)

### Computational Studies

### Example 7: Molecular Docking Studies

Methods and Materials

Curcumin inhibits 26S proteasome activity by direct inhibition of dual-specificity tyrosine-regulated kinase 2 (DYRK2) and this target protein was deployed for docking studies. The docking results clearly interprets the most active conjugate of the six synthesized compounds are having better docking score. Obtaining a balanced pharmacokinetic (ADME—Absorption, Distribution, Metabolism, and Excretion) properties of drug-like molecule is one the most difficult and challenging part of the drug development process.

The execution of molecular docking study was to identify whether CMC compounds modulate T47D and to identify potential binding sites for well-established ER—breast cancer target (PDB ID:5ZTN). Prediction of binding sites was performed by a combinatorial analysis. Binding site prediction was done by conducting literature reviews on DYRK2 target. Computational tools such as DoGSiteScorer and ScanProsite were used to predict the binding sites for the same. DoGSiteScorer reported a drug score of 81% having 41% of non-polar, 28% of polar, 18% of −ve and 13% of +ve amino acids and including 225 interaction points within the binding pocket. Validation of binding sites was carried out by establishing a comparative analysis of binding sites obtained from all three sources. Predicted binding sites for DYRK2 include Ile, Ala, Lys, Phe, Leu, and Asp involved in the key binding interactions.

Molecular docking studies were carried out by FlexX4, which exploits incremental construction algorithm for the prediction of dock score. The significance of the docking score implies how comfortable the ligand is interacting with the protein. Prediction of binding affinity and ligand efficiency (L.E) were performed by HYDE algorithm. Chain A of protein was considered for docking study since the amino acid residues present in the binding site were associated with chain A. Top 100 poses of solutions were generated by considering three different stereo modes of ligands such as E/Z, R/S and pseudo R/S. Binding of ligand to protein was driven by the enthalpy-entropy based hybrid approach.

Results

Even though curcumin has a good docking score and comfortably binds to the pocket of the protein target, the compound is not stable while considering desolvation terms and torsional alerts. On the other hand, CMC 2 compound has acceptable docking scores along with free binding affinity in agreement with desolvation terms and torsional alerts. Docking analysis revealed the selectivity of interactions with key amino acids, surface characteristics including the regulatory mechanism of the DYRK2. To better characterize and to make decisions on drug-like derivatives, pharmacokinetic studies to predict few ADME properties to understand the liability was carried out. CMC 2 compound showed optimally balanced properties of aqueous solubility (Sol), HERG liability (HERG II), developmental toxicity (Dev. Tox.), P-gycoprotein substrate/non-substrate (P-gp) and 2D6 isoform of P450 affinity data. The violation of drug-likeness, Lipinski rule including oral bioavailability could be overcome by lead optimization methods to design derivatives within the applicability domain of potency and all pharmacokinetic properties. The predicted ADME data looks promising (Table 3). Even though the oral administered animal studies gave us the preliminary results, the blood serum of the treated animal at different intervals of time to analyze the presence of our conjugate and or the hydrolyzed products will be investigated. Further, Predicted ADME properties of CMC compounds were given in Table 4.

TABLE 4
Predicted ADME properties of CMC compounds

| | | Aq. Sol | HERG | | | | |
|---|---|---|---|---|---|---|---|
| | | (log | II | Dev | CYP2D6 | P-gp | HIA |
| Name | Log P | mol/L) | Inhibitor | Tox | substrate | substrate | % |
| CUR | 3.852 | −3.878 | + | + | Med | − | 84.38 |
| CMC1 | 5.859 | −4.644 | − | − | Low | − | 81.65 |
| CMC 2 | 4.092 | −4.031 | − | − | Low | + | 66.25 |
| CMC 3 | 4.869 | −4.010 | − | − | Low | + | 68.18 |
| CMC 4 | 4.872 | −3.700 | − | + | Low | + | 61.82 |
| CMC 5 | 7.314 | −2.981 | + | + | Med | − | 81.50 |
| CMC 6 | 5.65 | −3.336 | − | + | Med | + | 67.89 |

In vitro studies confirmed the significant role of CMC 2 compound in eliciting anti-cancer activity. In silico studies conducted on synthesized hybrid conjugates and reported the binding affinity, significant interactions as well as bioavailability of these novel compounds with respect to curcumin. Out of 6 hybrid conjugates, CMC 2 compound exhibited higher dock score, binding energy as well as ligand efficiency. The binding energy of curcumin was found to be −24 kJ/mol, ligand efficiency 0.22, and dock score of −29.24. But CMC 2 compound exhibited a much higher range of these parameters, which indicates the likeliness of this compound to inhibit DYRK2. Even though the docking score of CMC 6 compound is considerably low, binding energy and ligand efficiency are comparable to CMC 2 compound. All the conjugates showed significant interactions with DYRK2. The comparative analysis of binding interactions revealed the presence of H-bonds with two significant amino-acid residues Leu231 and Asp295 in all the derivatives. NH— group of Leu231 made H-bond interactions with the protein, while polar amino acid Asp295 contributes in making stronger interactions with target protein by donating hydrogen atoms.

Bioavailability studies emphasize on the significance of human intestinal absorption, affinity towards P450 isoform CYP2D6, developmental toxicity, hERG inhibition, and lipophilicity. Affinity towards P450 isoform confirms the metabolic stability of compounds. Low/medium range of affinity is acceptable since a higher affinity towards cytochrome P450 results in the decreased therapeutic value of lead-like compounds. This is due to the higher rate of conversion of compounds into metabolic end products before eliciting its therapeutic activity (Priest et al., Channels 2008, 87, 87-93). Developmental toxicity is highly undesirable since this could affect the entire homeostasis process. hERG is a gene encoding alpha subunit of potassium ion channel. Drug-induced inhibition of hERG results in the development of cardiac-related disorders (Zhang et al., Acta Pharm. Sinica B 2018, 8, 721-732). Lipophilicity is an essential parameter depicting the permeability of lead-like molecules into biological membranes.

Curcumin reported for anti-cancer activity was found to inhibit hERG and possesses developmental toxicity, which is not appreciable. But the hybrid conjugate CMC 2 compound has got the optimal balance for all the above-mentioned parameters. Hence, potency of CMC 2 compound in executing anti-cancer activity is confirmed by in-vitro and in-silico approaches. CMC 3 compound showed good bioavailability scores which are comparable to CMC 2 compound. All conjugates exhibited good intestinal absorption profiles, metabolic profiles, and lipophilicity. But CMC 4, CMC 5, and CMC 6 compounds were found to exhibit developmental toxicity and CMC 5 compound was reported for hERG inhibition.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A curcumin conjugate comprising the general formula 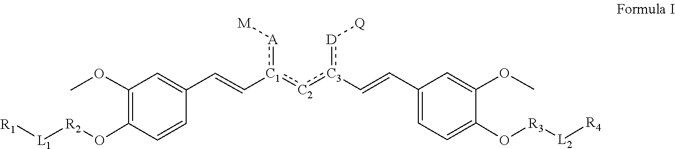
wherein the dotted lines between A and C₁, C₁ and C₂, C₂ and C₃ and C₃ and D indicate that a single or double bond may be present, as valence permits,
wherein the dotted lines A and M, and D and Q indicate that a single bond or no bond may be present, as valence permit,
wherein C₁, C₂, and C₃ are carbon atoms,
wherein A and D are oxygen atoms,
wherein M and Q are independently absent, or hydrogen, as valence permits,
wherein R₂ and R₃ can be independently absent, one or more amino acids or salts thereof, nucleic acids, lipids, polysaccharides, polymers, substituted or unsubstituted carbonyl groups, alkyl groups, alkenyl groups, alkynyl groups, aryl groups, heteroaryl groups, or other organic groups containing between C₁ and C₃₀ carbon atoms, inclusive, preferably between C₁ and C₂₀ carbon atoms, inclusive, more preferably between C₁ and C₁₀ carbon atoms, with the proviso that at least one of R₂ or R₃ is present,
wherein R₁ and R₄ can be independently absent, one or more amino acids or salts thereof, nucleic acids, lipids, polysaccharides, polymers, substituted or unsubstituted halogen groups, alkyl groups, alkenyl groups, alkynyl groups, aryl groups, heteroaryl groups, organic protecting groups, small molecules, or other organic groups containing between C₁ and C₃₀ carbon atoms, inclusive, preferably between C₁ and C₂₀ carbon atoms, inclusive, more preferably between C₁ and C₁₀ carbon atoms, and
wherein L₁ and L₂ can be independently absent, substituted or unsubstituted amide groups, halogen groups, alkyl groups, alkenyl groups, alkynyl groups, aryl groups, heteroaryl groups, or other organic groups containing between C₁ and C₂₀ carbon atoms, inclusive, preferably between C₁ and C₁₀ carbon atoms, inclusive or a combination thereof or pharmaceutically acceptable salt(s), polymorph(s), solvent(s), hydrate(s), crystal forms, and/or enantiomer(s) thereof.

2. The curcumin conjugate of claim 1, wherein the curcumin is in the keto form, enol form or combinations thereof.

3. The curcumin conjugate of claim 1, wherein R₁ and R₄ are each independently substituted or unsubstituted halogen groups.

4. The curcumin conjugate of claim 1, wherein R₁ and R₄ are each independently dichloroacetic acid.

5. The curcumin conjugate of claim 1, wherein M and Q are absent, the bond between A and C₁, and D and C₃ are double bonds, and the bonds between C₁ and C₂, and C₂ and C₃ are single bonds.

6. The curcumin conjugate of claim 1, wherein (i) the bond between C₁ and A is a double bond, M is absent, the bond between C₁ and C₂ is a single bond, the bond between C₂ and C₃ is a double bond, the bond between C₃ and D is a single bond, and Q is hydrogen, or (ii) the bond between C₃ and D is a double bond, Q is absent, the bond between C₂ and C₃ is a single bond, the bond between C₁ and C₂ is a double bond, the bond between C₁ and A is a single bond, and M is hydrogen.

7. The curcumin conjugate of claim 1, wherein R₂ and R₃ are one or more amino acids or salts thereof.

8. The curcumin conjugate of claim 1, wherein R₂ and R₃ are each independently substituted or unsubstituted carbonyl groups.

9. The curcumin conjugate of claim 1, wherein L₁ and L₂ are each independently substituted or unsubstituted alkyl groups.

10. The curcumin conjugate of claim 1, wherein L₁ and L₂ are each independently substituted or unsubstituted amide groups.

11. The curcumin conjugate of claim 1, wherein the curcumin conjugate is selected from the group consisting of the structure of any one of the following compounds: 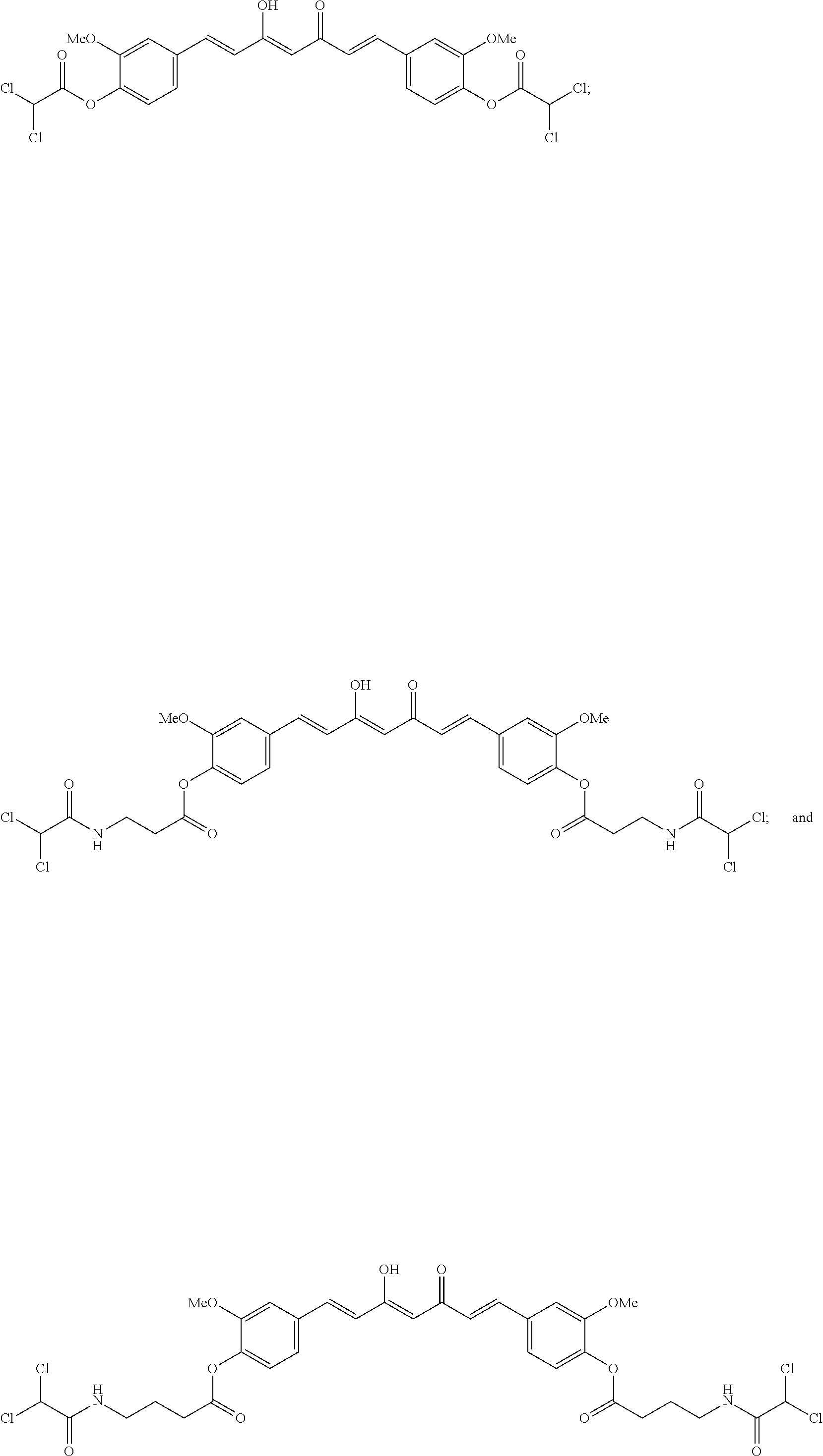 [SMILES: COc1cc(/C=C/C(=O)/C=C(O)/C=C/c2ccc(OC(=O)C(Cl)Cl)c(OC)c2)ccc1OC(=O)C(Cl)Cl ; COc1cc(/C=C/C(=O)/C=C(O)/C=C/c2ccc(OC(=O)CCNC(=O)C(Cl)Cl)c(OC)c2)ccc1OC(=O)CCNC(=O)C(Cl)Cl ; "and" ; COc1cc(/C=C/C(=O)/C=C(O)/C=C/c2ccc(OC(=O)CCCNC(=O)C(Cl)Cl)c(OC)c2)ccc1OC(=O)CCCNC(=O)C(Cl)Cl]
or pharmaceutically acceptable salt(s), polymorph(s), solvent(s), hydrate(s), crystal forms, and/or enantiomer(s) thereof.

12. A pharmaceutical composition comprising an effective amount of at least one of the curcumin conjugates of claim 1.

13. The pharmaceutical composition of claim 12, further comprising a pharmaceutically acceptable excipient.

14. The pharmaceutical composition of claim 12, wherein the composition is formulated for oral delivery.

15. The pharmaceutical composition of claim 12, wherein the composition is optically pure.

16. A method treating a cancer in a subject in need thereof comprising administering to the subject a therapeutically effective amount of the curcumin conjugate of claim 1 or a pharmaceutical composition thereof.

17. The method of claim 16, wherein the cancer is selected from squamous cell carcinoma, small-cell lung cancer, non-small cell lung cancer (NSCLC), lung adenocarcinoma, squamous cell lung cancer, peritoneum cancer, hepatocellular cancer, stomach cancer, tastrointestinal cancer; esophageal cancer, pancreatic cancer; glioblastoma, cervical cancer, ovarian cancer, liver cancer, bladder cancer, breast cancer, colon cancer, rectal cancer, colorectal cancer, endometrial cancer, uterine cancer, salivary gland carcinoma, renal cancer, prostate cancer, vulval cancer, thyroid cancer, hepatocellular carcinoma (HCC), anal carcinoma, penile carcinoma, or head and neck cancer.

18. The method of claim 17, wherein the cancer is breast cancer.

19. The method of claim 16, wherein the subject is selected from the group consisting of mammal, human or genetically engineered mouse (GEM).

20. A method inhibiting cancer cell growth in a subject in need thereof comprising administering to the subject a therapeutically effective amount of the curcumin conjugate of claim 1 or a pharmaceutical composition thereof.
